(19) United States
(12) Patent Application Publication
(10) Pub. No.: US 2023/0256045 A1
(43) Pub. Date: Aug. 17, 2023

(54) COMPOSITIONS AND METHODS FOR THE PREVENTION OF TREATMENT OF HUMN PAPILLOMA VIRUS (HPV)-ASSOCIATED DISEASE

(71) Applicant: Petros TYMPANIDIS, Athens (GR)

(72) Inventor: Petros TYMPANIDIS, Athens (GR)

(21) Appl. No.: 17/951,329

(22) Filed: Sep. 23, 2022

Related U.S. Application Data
(60) Provisional application No. 63/247,356, filed on Sep. 23, 2021.

(51) Int. Cl. A61K 36/63; A61K 36/185; A61K 36/53; A61P 31/20; A61K 9/06

(52) U.S. Cl. CPC A61K 36/63; A61K 36/185; A61K 36/53; A61P 31/20; A61K 9/06

(57) ABSTRACT

Aspects described herein relate to pharmaceutical compositions comprising Saint John's Wort, olive oil, and thyme for the prevention or treatment of human papilloma virus (HPV)-associated disease in a subject in need thereof, particularly HPV-induced micro lesions and/or dryness of the cervical-vaginal mucosa. The pharmaceutical compositions are also provided for inhibiting viral replication and viral production of HPV 16 in a subject in need thereof. Methods of use are also provided, along with kits and containers for packaging the disclosed compositions together with instructions for their use.

### CROSS-REFERENCE TO RELATED APPLICATIONS

The present invention is a U.S. Utility patent application that claims priority to U.S. Provisional Patent Application No. 63/247,356, filed on Sep. 23, 2021, the entire disclosure of which is incorporated herein by reference.

### TECHNICAL FIELD

Aspects described herein relate to compositions and methods for the prevention or treatment of human papilloma virus (HPV)-associated disease, particularly to compositions comprising Saint John's Wort, olive oil, and thyme for the prevention or treatment of HPV-induced micro lesions and/or dryness of the cervical-vaginal mucosa and inhibition of viral replication and viral production for HPV 16.

### BACKGROUND

Papillomaviruses are small nonenveloped DNA viruses with a virion size of about 55 nm in diameter. More than 100 human papilloma virus (HPV) genotypes are completely characterized, and a higher number is presumed to exist. HPV is a known cause of cervical cancers, as well as some vulvar, vaginal, penile, oropharyngeal, anal, and rectal cancers. Although most HPV infections are asymptomatic and clear spontaneously, persistent infections with one of the oncogenic HPV types can progress to precancer or cancer. Other HPV-associated diseases can include common warts, plantar warts, flat warts, anogenital warts, anal lesions, epidermodysplasia, focal epithelial hyperplasia, mouth papillomas, verrucous cysts, laryngeal papillomatosis, squamous intraepithelial lesions (Sits), cervical intraepithelial neoplasia (CIN), vulvar intraepithelial neoplasia (VIN), and vaginal intraepithelial neoplasia (VAIN).

Many of the known HPV types cause benign lesions with a subset being oncogenic. Based on epidemiologic and phylogenetic relationships, HPV types are classified into fifteen “high risk types” (HPV 16, 18, 31, 33, 35, 39, 45, 51, 52, 56, 58, 59, 68, 73, and 82) and three “probable high risk types” (HPV 26, 53, and 66), which together are known to manifest as low and high grade cervical changes and cancers, as well as other anogential cancers such as vulval, vaginal, penile, anal, and perianal cancer, as well as head and neck cancers. Recently, the association of high risk types HPV 16 and 18 with breast cancer was also described. Eleven HPV types classified as “low risk types” (HPV 6, 11, 40, 42, 43, 44, 54, 61, 70, 72, and 81) are known to manifest as benign low-grade cervical changes, genital warts and recurrent respiratory papillomatosis. Cutaneous HPV types 5, 8, and 92 are associated with skin cancer. In some HPV-associated cancers, the immune system is depressed and correspondingly, the antitumor response is significantly impaired (See, e.g., Suresh & Burtness (2017) Am. J. Hematol. Oncol. 13(6):20-27).

In particular, HPV 16 and HPV 18 are responsible for about 70% of all cervical cancers worldwide. To date, two prophylactic HPV vaccines against these HPV types are on the market (Gardasil®9 and Cervarix®. The aim of these prophylactic vaccines is to induce humoral immune responses by stimulating the production of neutralizing antibodies specific for the HPV viral capsid proteins, L1 and L2. Although these preventive vaccines are an important milestone for the control of HPV induced cervical cancer and possibly other HPV-associated malignancies, the effect of these vaccines will not be significantly observed for 20-40 years (Ma et al. (2010) Current Cancer Therapy Reviews, 6(2):81-103). Furthermore, the high cost, issues with social acceptance, and limitations in health care systems through which the vaccine can be provided limit the availability of this vaccine, particularly in developing countries where HPV-associated anogenital cancers are most commonly found.

Accordingly, there is a continuing need for safe and effective compositions and methods for the prevention or treatment of HPV-associated disease.

### SUMMARY

To address the foregoing problems, in whole or in part, and/or other problems that may have been observed by persons skilled in the art, the present disclosure provides compositions and methods as described by way of example as set forth below.

A pharmaceutical composition is provided for the prevention or treatment of human papilloma virus (HPV)-associated disease in a subject in need thereof, comprising a pharmaceutically acceptable carrier and active agents comprising: Saint John's Wort, olive oil, and thyme; wherein the active agents are each present in therapeutically effective amounts to prevent HPV-associated disease in the subject. In certain embodiments, the Saint John's Wort is in an amount of about 0.2 mg±1.5 mg, the olive oil is in an amount of about 1.0 mg±1.5 mg, and the thyme is in an amount of about 0.2 mg±1.5 mg.

In particular embodiments, the pharmaceutical composition is formulated as a vaginal cream, ointment, or gel. In other embodiments, the HPV-associated disease comprises HPV-induced micro lesions and/or dryness of the cervical-vaginal mucosa. In other embodiments, the pharmaceutical composition inhibits viral replication and viral production of HPV 16.

In some embodiments, a method is provided for the prevention or treatment of HPV-associated disease in a subject in need thereof, comprising administering to the subject a pharmaceutical composition comprising a pharmaceutically acceptable carrier and active agents comprising: Saint John's Wort, olive oil, and thyme; wherein the active agents are each present in therapeutically effective amounts to prevent or treat HPV-associated disease in the subject. In certain embodiments, the Saint John's Wort is in an amount of about 0.2 mg±1.5 mg, the olive oil is in an amount of about 1.0 mg±1.5 mg, and the thyme is in an amount of about 0.2 mg±1.5 mg.

In particular embodiments, administration of the pharmaceutical composition is initiated after the subject's menses, is administered daily for a month followed by administration every other day for 2 to 6 months, further wherein the administration of the pharmaceutical composition is paused during the subject's menses. In further embodiments, the pharmaceutical composition within the methods is formulated as a vaginal cream, ointment, or gel. In other embodiments, the HPV-associated disease comprises HPV-induced micro lesions and/or dryness of the cervical-vaginal mucosa. In other embodiments, administration of the pharmaceutical composition inhibits viral replication and viral production of HPV 16.

In some embodiments, a kit is provided for the prevention or treatment of HPV-associated disease in a subject in need thereof comprising a pharmaceutical composition and instructions for the administration of the pharmaceutical composition in a manner effective to treat HPV-associated disease in the subject, wherein the pharmaceutical composition comprises a pharmaceutically acceptable carrier and active agents comprising: Saint John's Wort, olive oil, and thyme; wherein the active agents are each present in therapeutically effective amounts to prevent or treat HPV-associated disease in the subject. In certain embodiments, the Saint John's Wort is in an amount of about 0.2 mg±1.5 mg, the olive oil is in an amount of about 1.0 mg±1.5 mg, and the thyme is in an amount of about 0.2 mg±1.5 mg.

In particular embodiments of the kit of the present invention, the instructions for the administration of the pharmaceutical composition describe initiating administration of the pharmaceutical composition after the subject's menses, administering the pharmaceutical composition daily for a month followed by administration every other day for 2 to 6 months, and further wherein the administration of the pharmaceutical composition is paused during the subject's menses. In further embodiments, the pharmaceutical composition within the kits is formulated as a vaginal cream, ointment, or gel. In other embodiments, the HPV-associated disease comprises HPV-induced micro lesions and/or dryness of the cervical-vaginal mucosa. In other embodiments, the kit and pharmaceutical composition are used to inhibit viral replication and viral production of HPV 16.

Other compositions, methods, features, and advantages of the invention will be or will become apparent to one with skill in the art upon examination of the following figures and detailed description. It is intended that all such additional compositions, methods, features, and advantages be included within this description, be within the scope of the invention, and be protected by the accompanying claims.

### BRIEF DESCRIPTION OF THE DRAWINGS

The invention can be better understood by referring to the following figures. The components in the figures are not necessarily to scale, emphasis instead being placed upon illustrating the principles of the invention.

FIG. 1 shows an evaluation of the toxicity of a pharmaceutical composition (“Benostan Cream”) to HPV-16 organotypic tissues. H&E staining is shown of tissue sections from Benostan Cream-treated persistently infected HPV-16 organotypic tissues that were grown for a total of 12 days. During this growth period the organotypic tissue was treated with Benostan Cream diluted 1:5 in tissue growth media for different durations. (A) days 0-12, (B) days 0-6, (C) days 7-12, (D) days 7-8, (E) days 9-10, (F) days 11-12, (G) control-PBS diluted 1:15 in tissue growth media, and (H) control-tissue growth media only.

FIG. 2 shows the effects of Benostan Cream on HPV-16 replication in organotypic tissue. H&E staining is shown of tissue sections from Benostan Cream-treated persistently infected HPV-16 organotypic tissues, representative of tissues used to measure the inhibitory effect of Benostan Cream on HPV-16 replication (see Table 1). Tissues were treated with Benostan Cream during days 7-12 exclusively of a total 12 days of tissue growth. (A) Benostan Cream diluted 1:5 in tissue growth media, (B) Benostan Cream diluted 1:10 in tissue growth media, (C) Benostan Cream diluted 1:15 in tissue growth media, (D) control-PBS diluted 1:5 in tissue growth media, and (E) control-tissue growth media only.

### DETAILED DESCRIPTION

The subject matter of the present invention now will be described more fully hereinafter with reference to the accompanying drawings, in which some, but not all embodiments of the subject matter of the present invention are shown. Like numbers refer to like elements throughout. The subject matter of the present invention may be embodied in many different forms and should not be construed as limited to the embodiments set forth herein; rather, these embodiments are provided so that this disclosure will satisfy applicable legal requirements. Indeed, many modifications and other embodiments of the subject matter of the present invention set forth herein will come to mind to one skilled in the art to which the subject matter of the present invention pertains having the benefit of the teachings presented in the foregoing descriptions and the associated drawings. Therefore, it is to be understood that the subject matter of the present invention is not to be limited to the specific embodiments disclosed and that modifications and other embodiments are intended to be included within the scope of the appended claims.

The present invention relates to compositions and methods for the prevention or treatment of HPV-associated disease, particularly to compositions comprising Saint John's Wort, olive oil, and thyme for the prevention or treatment of HPV-induced micro lesions and/or dryness of the cervical-vaginal mucosa. In other embodiments, the compositions and methods inhibit viral replication and viral production of HPV 16.

HPV infections represent one of the most common sexually transmitted infections and are primarily transmitted through vaginal, anal, or oral sex with someone who has the virus. HPV can be passed even when an infected person has no signs or symptoms, and symptoms of HPV can develop years after sexual contact with someone who is infected.

As described in the background section herein, Papilloma viruses are small nonenveloped DNA viruses with a virion size of about 55 nm in diameter (virions are the mature forms of the virus). More than 100 human papilloma virus (HPV) genotypes are completely characterized, and a higher number is presumed to exist. Based on epidemiologic and phylogenetic relationships, HPV types are classified into fifteen “high risk types” (HPV 16, 18, 31, 33, 35, 39, 45, 51, 52, 56, 58, 59, 68, 73, and 82) and three “probable high risk types” (HPV 26, 53, and 66), which together are known to manifest as low and high grade cervical changes and cancers, as well as other anogential cancers such as vulval, vaginal, penile, anal, and perianal cancer, as well as head and neck cancers. Eleven HPV types classified as “low risk types” (HPV 6, 11, 40, 42, 43, 44, 54, 61, 70, 72, and 81) are known to manifest as benign low-grade cervical changes, genital warts and recurrent respiratory papillomatosis.

Without being bound by any particular theory or mechanism of action, it is thought that the combination of active agents of the present invention treats HPV-associated disease in a subject in need thereof by helping to restore normal vaginal conditions after colposcopy or treatment of warts in the vagina. This unique combination of active agents restores the physiological environment intra-vaginally by providing relief and smooth conditions for rapid healing. When formulated as a cream, gel, or the like, the pharmaceutical composition creates a protective film in the vaginal mucosa that acts as a defensive barrier and provides optimal conditions for the healing of HPV-induced epithelial micro lesions and/or dryness of the cervical-vaginal mucosa. The pharmaceutical composition repairs and re-epithelizes lesions of the cervical-vaginal mucosa, prevents the risk of HPV-induced lesions, rebalances vaginal microbiota, improves vaginal health, and creates conditions for rapid healing of lesions caused by scratching due to burning and pruritus. In other embodiments, the pharmaceutical composition inhibits viral replication and viral production of HPV 16.

### I. Pharmaceutical Compositions

In one embodiment, the present invention provides a pharmaceutical composition for the prevention or treatment of HPV-associated disease in a subject in need thereof, comprising a pharmaceutically acceptable carrier and active agents comprising: Saint John's Wort, olive oil, and thyme; wherein the active agents are each present in therapeutically effective amounts to prevent HPV-associated disease in the subject. In particular embodiments, the HPV-associated disease comprises HPV-induced micro lesions and/or dryness of the cervical-vaginal mucosa. In other embodiments, the pharmaceutical composition inhibits viral replication and viral production of HPV 16.

St. John's Wort (Hypericum perforatum) is a medicinal plant with antiviral, anti-inflammatory and anti-pain activities (Birt, D. F. et al. (2009) Pharmaceutical biology, 47(8):774-782). In certain embodiments, the Saint John's Wort within the pharmaceutical composition of the invention is in an amount of about 0.2 mg±1.5 mg.

Olive oil is derived from olive plants. Several species within the olive family, botanically known as Olea europaea, provide commercial products such as food, lumber, cosmetics and medicine (Omar S. H. (2010). Scientia pharmaceutica, 78(2):133-154). In certain embodiments, the olive oil within the pharmaceutical composition of the invention is in an amount of about 1.0 mg±1.5 mg.

Thyme (Thymus vulgaris) is medicinal plant with anti-infective, cardioprotective, gastroprotective, anti-inflammatory, and immunomodulatory activities (Stahl, B. E. & Venskutonis, R. P. (2012). Thyme. K. V. Peter (Ed.), Handbook of Herbs and Spices (second ed.), Woodhead Publishing, London, UK, pp. 499-525). In certain embodiments, the thyme within the pharmaceutical composition of the invention is in an amount of about 0.2 mg±1.5 mg.

The active agents of the present invention are effective over a wide dosage range. For example, in treating adult humans, pharmaceutical compositions and methods of the present invention comprise therapeutically effective dosages of Saint John's Wort, olive oil, and thyme from 0.01 mg to 1,000 mg, from 0.5 mg to 500 mg, from 1 mg to 100 mg, from 5 mg to 50 mg, and from 10 mg to 25 mg. Alternatively, in treating adult humans, pharmaceutical compositions and methods of the present invention comprise dosages of Saint John's Wort, olive oil, and thyme of 0.01 mg, 0.05 mg, 0.1 mg, 0.25 mg, 0.5 mg, 0.75 mg, 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1,000 mg.

In other embodiments, the therapeutically effective dose of Saint John's Wort, olive oil, and thyme within the pharmaceutical compositions and methods of the invention typically ranges from about 1 μg/kg to about 500 mg/kg, about 10 μg/kg to about 500 mg/kg, about 100 μg/kg to about 500 mg/kg, about 1 mg/kg to about 500 mg/kg, about 1 mg/kg to about 400 mg/kg, about 1 mg/kg to about 300 mg/kg, about 1 mg/kg to about 200 mg/kg, about 1 mg/kg to about 100 mg/kg, about 1 mg/kg to about 75 mg/kg, about 1 mg/kg to about 50 mg/kg, or about 1 mg/kg to about 25 mg/kg. In another embodiment, the dosage of Saint John's Wort, olive oil, and thyme is an amount of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 110 mg/kg, about 120 mg/kg, about 125 mg/kg, about 130 mg/kg, about 140 mg/kg, about 150 mg/kg, about 160 mg/kg, about 170 mg/kg, about 175 mg/kg, about 180 mg/kg, about 190 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 425 mg/kg, about 450 mg/kg, about 475 mg/kg, to about 500 mg/kg.

For particular agents with greater toxicity profiles, one of skill in the art will appreciate that the therapeutically effective amount may be even lower to achieve a nontoxic but therapeutically effective dose, for example from about 1 μg/kg to about 1 μg/kg, about 50 pg/kg to about 1 μg/kg, about 100 μg/kg to about 1 μg/kg, about 500 μg/kg to about 1 μg/kg, about 1 ng/kg to about 1 mg/kg, about 50 ng/kg to about 1 mg/kg, about 100 ng/kg to about 1 mg/kg, about 500 ng/kg to about 1 mg/kg, about 1 μg/kg to about 1 mg/kg, about 50 μg/kg to about 1 mg/kg, about 100 μg/kg to about 1 mg/kg, or about 500 μg/kg to about 1 mg/kg. In such embodiments, the therapeutically effective dose of Saint John's Wort, olive oil, and thyme is an amount of about 1 pg/kg, about 5 pg/kg, about 10 pg/kg, about 20 pg/kg, about 30 pg/kg, about 40 pg/kg, about 50 pg/kg, about 100 pg/kg, about 200 pg/kg, about 300 pg/kg, about 400 pg/kg, about 500 pg/kg, about 600 pg/kg, about 700 pg/kg, about 800 pg/kg, about 900 pg/kg, about 1 ng/kg, about 5 ng/kg, about 10 ng/kg, about 20 ng/kg, about 30 ng/kg, about 40 ng/kg, about 50 ng/kg, about 100 ng/kg, about 200 ng/kg, about 300 ng/kg, about 400 ng/kg, about 500 ng/kg, about 600 ng/kg, about 700 ng/kg, about 800 ng/kg, about 900 ng/kg, about 1 μg/kg, about 5 μg/kg, about 10 μg/kg, about 20 μg/kg, about 30 μg/kg, about 40 μg/kg, about 50 μg/kg, about 100 μg/kg, about 200 μg/kg, about 300 μg/kg, about 400 μg/kg, about 500 μg/kg, about 600 μg/kg, about 700 μg/kg, about 800 μg/kg, about 900 μg/kg, about 1 mg/kg, and other such values between about 1 pg/kg and about 1 mg/kg.

In other embodiments, the concentration of Saint John's Wort, olive oil, and thyme within the pharmaceutical compositions and methods of the present invention may also be expressed as a percent of the active agent by weight. In one embodiment, the amount of Saint John's Wort, olive oil, and thyme may range from about 0.0001% to about 20% by weight. In another embodiment, the amount may range from about of 1% to about 15% by weight. In yet another embodiment, the amount may range from 3.75% to about 10% by weight. In another embodiment, the amount may range from about 1% to about 99% by weight, from about 10% to about 80% by weight, and more typically, from about 20% to about 60% by weight. In another embodiment, the amount may be about 5% by weight, less than about 5% by weight, less than about 4% by weight, less than about 3% by weight, less than about 2% by weight, or less than about 1% by weight. In another embodiment, the amount may be greater than about 5%, greater than about 10%, greater than about 15%, greater than about 20%, greater than about 25%, greater than about 30%, greater than about 35%, greater than about 40%, greater than about 50%, greater than about 55%, greater than about 60%, greater than about 65%, greater than about 70%, or greater than about 75%.

It is contemplated that the pharmaceutical compositions of the present invention can include any ingredient (e.g., active agent, carrier, etc.) described throughout this specification, or any combination thereof. The concentrations of the any ingredient within the pharmaceutical compositions can vary. In non-limiting embodiments, for example, the pharmaceutical compositions can comprise, consist essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight or volume of the total composition. A person of ordinary skill in the art would understand that the concentrations can vary depending on the addition, substitution, and/or subtraction of ingredients in a given composition.

As used herein, the term“pharmaceutically acceptable carrier” includes solvents, dispersion media, coatings, antibacterial and antifungal agents, isotonic and absorption delaying agents, and the like, compatible with pharmaceutical administration.

As one of ordinary skill in the art will appreciate, the presently disclosed pharmaceutical compositions may be formulated to be compatible with their intended route of administration (e.g., oral, transdermal, sublingual, and parenteral, including intraperitoneal, intravenous, subcutaneous, or intramuscular administration). Suitable formulations and their appropriate carrier vehicles are described, for example, in Remington's The Science and Practice of Pharmacy (23^{rd} ed.; Elsevier Publishing Company, Academic Press, 2021), incorporated herein by reference.

In particular embodiments, the pharmaceutical composition is formulated for transmucosal administration. For transmucosal administration, penetrants appropriate to the barrier to be permeated are used in the composition. Such penetrants are generally known in the art, and include, for example, detergents, bile salts, and fusidic acid derivatives. Transmucosal administration can also be accomplished through the use of sprays or suppositories. For transmucosal, the active compounds may be formulated into ointments, salves, gels, or creams as generally known in the art.

Accordingly, pharmaceutical compositions of the present invention may be formulated as a vaginal cream, gel, ointment, paste, foam, spray, suppository, pessary, tampon, tablet, or ring, in which the active agents are combined with one or more conventional nontoxic carriers and/or excipients suitable for vaginal administration. The vaginal formulations of the present invention can be manufactured using conventional processes as disclosed in Remington's The Science and Practice of Pharmacy (23^{rd} ed.; Elsevier Publishing Company, Academic Press, 2021)(see also active agent formulations as adapted in U.S. Pat. Nos. 6,515,198; 6,500,822; 6,417,186; 6,416,779; 6,376,500; 6,355,641; 6,258,819; 6,172,062; and 6,086,909). The vaginal formulation can be fabricated to disintegrate rapidly or over a period of several hours.

In particular embodiments, the pharmaceutical compositions of the present invention are formulated as creams, ointments, or gels, and/or may be prepared so as to contain liposomes, micelles, and/or microspheres.

Creams, as is well known in the art of pharmaceutical formulation, are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum active agent delivery, and, preferably, will provide for other desired characteristics as well, e.g., emolliency or the like. As with other carriers or vehicles, an ointment base should be inert, stable, nonirritating and nonsensitizing. As explained in Remington's The Science and Practice of Pharmacy (23^{rd} ed.; Elsevier Publishing Company, Academic Press, 2021), ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophihc petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Gels, as will be appreciated by those working in the field of pharmaceutical formulations, are semisolid, suspension-type systems. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol® trademark. Also preferred are hydrophihc polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methylcellulose; gums such as tragacanth and xanthan gum; sodium alginate; and gelatin, h order to prepare a uniform gel, dispersing agents such as alcohol or glycerin can be added, or the gelling agent can be dispersed by trituration, mechanical mixing, and/or stirring.

Other components may also be incorporated into the pharmaceutical compositions described herein. For example, solubilizers may be used to solubilize certain active agents. For those active agents having an unusually low rate of permeation through the skin or mucosal tissue, it may be desirable to include a permeation enhancer in the formulation; suitable enhancers are as described elsewhere herein. Additional components may also include, but are not limited to, stiffening agents, antioxidants, preservatives, and the like.

The presently disclosed compositions also can be prepared with carriers that will protect the active agents against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

### II. Methods

In some embodiments, a method is provided for the prevention or treatment of HPV-associated disease in a subject in need thereof, comprising administering to the subject a pharmaceutical composition comprising a pharmaceutically acceptable carrier and active agents comprising: Saint John's Wort, olive oil, and thyme; wherein the active agents are each present in therapeutically effective amounts to prevent or treat HPV-associated disease in the subject. In certain embodiments, the Saint John's Wort is in an amount of about 0.2 mg±1.5 mg, the olive oil is in an amount of about 1.0 mg±1.5 mg, and the thyme is in an amount of about 0.2 mg±1.5 mg. In particular embodiments, the HPV-associated disease comprises HPV-induced micro lesions and/or dryness of the cervical-vaginal mucosa. In other embodiments, administration of the pharmaceutical composition inhibits viral replication and viral production of HPV 16.

In particular embodiments, administration of the pharmaceutical composition is initiated after the subject's menses, is administered daily for a month followed by administration every other day for 2 to 6 months, further wherein the administration of the pharmaceutical composition is paused during the subject's menses. In further embodiments, the pharmaceutical composition within the methods is formulated as a vaginal cream, ointment, or gel.

### III. Kits and Containers

In some embodiments, a kit is provided for the prevention or treatment of HPV-associated disease in a subject in need thereof comprising a pharmaceutical composition and instructions for the administration of the pharmaceutical composition in a manner effective to treat HPV-associated disease in the subject, wherein the pharmaceutical composition comprises a pharmaceutically acceptable carrier and active agents comprising: Saint John's Wort, olive oil, and thyme; wherein the active agents are each present in therapeutically effective amounts to prevent or treat HPV-associated disease in the subject. In certain embodiments, the Saint John's Wort is in an amount of about 0.2 mg±1.5 mg, the olive oil is in an amount of about 1.0 mg±1.5 mg, and the thyme is in an amount of about 0.2 mg±1.5 mg.

In particular embodiments of the kit of the present invention, the instructions for the administration of the pharmaceutical composition describe initiating administration of the pharmaceutical composition after the subject's menses, administering the pharmaceutical composition daily for a month followed by administration every other day for 2 to 6 months, and further wherein the administration of the pharmaceutical composition is paused during the subject's menses. In further embodiments, the pharmaceutical composition within the kits is formulated as a vaginal cream, ointment, or gel. In other embodiments, the HPV-associated disease comprises HPV-induced micro lesions and/or dryness of the cervical-vaginal mucosa. In other embodiments, the kit and pharmaceutical composition are used to inhibit viral replication and viral production of HPV 16.

The kit may be a packaged kit comprising a container, preferably sealed, for housing the pharmaceutical composition during storage and prior to use, and instructions for carrying out administration in a manner effective to prevent or treat HPV-associated disease. The container can be a bottle, dispenser, or package.

The instructions will typically be written instructions on a package insert and/or on a label. The kit may also include a device for administering the pharmaceutical composition. The pharmaceutical composition may be any suitable formulation as described herein.

The kit may contain multiple formulations of different dosages of the same active agent or the kit may also contain multiple formulations of different active agents. The kit may contain multiple formulations of different dosages of the same active agent or the kit may also contain multiple formulations of different active agents.

The kit may contain formulations suitable for sequential, separate, and/or simultaneous use, and instructions for carrying out administration where the formulations are administered sequentially, separately, and/or simultaneously.

The parts of the kit may be independently held in one or more containers-such as bottles, syringes, cannulas, plates, wells, blister packs, or any other type of pharmaceutical packaging.

### IV. Definitions

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the subject matter of the present invention. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

As used herein, the term “subject” as treated by the presently disclosed compositions and methods is desirably a human subject, although it is to be understood that the methods described herein are effective with respect to all vertebrate species, which are intended to be included in the term “subject.” Accordingly, a “subject” can include a human subject for medical purposes (e.g., the diagnosis or treatment of an existing disease, disorder, or condition, or the prophylactic diagnosis or treatment for preventing the onset of a disease, disorder, or condition) or an animal subject for veterinary purposes. Suitable animal subjects include mammals, including, but not limited to, primates, bovines, ovines, caprines, porcines, equines, felines, canines, lagomorphs, and rodents. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

The term “effective amount,” as in “a therapeutically effective amount,” of a composition or active agent refers to the amount of the composition or active agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

Where the subject is a human subject, the dosage levels are based upon a body weight of approximately 70 kg. It will be understood, however, that the specific dose level and frequency of dosage for any particular subject can be varied so as to obtain an amount of the active agents effective to achieve the desired therapeutic response without being toxic to the subject. The selected dosage level will depend on a variety of factors including: body weight; age; general health and prior medical history; sex; diet; route, time, and duration of administration; length of action and metabolic stability of the administered active agent; rate of excretion of the administered active agent; drug combinations and/or materials used in combination with the particular active agent employed; and severity of HPV-associated disease. A physician having ordinary skill in the art can readily determine and prescribe the effective amount of the active agents within the presently disclosed pharmaceutical compositions and methods.

By “pharmaceutically acceptable,” such as in the recitation of a “pharmaceutically acceptable carrier,” or a “pharmaceutically acceptable acid addition salt,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. “Pharmacologically active” (or simply “active”) as in a “pharmacologically active” derivative or metabolite, refers to a derivative or metabolite having the same type of pharmacological activity as the parent compound. When the term “pharmaceutically acceptable” is used to refer to a derivative (e.g., a salt or an analog) of an active agent, it is to be understood that the compound is pharmacologically active as well, i.e., therapeutically effective for treating HPV-associated disease, in mammals, particularly humans. In other embodiments, the pharmaceutical composition inhibits viral replication and viral production of HPV 16.

### EXAMPLES

### Example 1

A pharmaceutical composition (“Benostan cream”) for the prevention or treatment of human papilloma virus (HPV)-associated disease was evaluated for activity against HPV-16.

### Materials and Methods

Established Cells and Virus: A HaCaT immortalized human keratinocyte cell line was provided to Pennsylvania State University (PSU) by Norbert Fusenig, DKFZ, Heidelberg. The cells were cultured in DMEM supplemented with 10% heat inactivated FBS, 25 μg/mL gentamicin and 0.11 mg/mL sodium pyruvate solution. The HPV-16 was propagated in an organotypic raft culture (Biryukov et al. (2015) Methods in Molecular Biology, 1249:317-331).

Evaluation of Toxicity by Benostan Cream to Organotypic Tissue: Benostan cream was evaluated at 1 concentration (1:5 dilution) for toxicity to the organotypic tissue. This was to assure that any inhibition of HPV replication was not merely the result of tissue death, which could be non-specific. All virus infected organotypic tissues were grown for a total of 12 days.

To determine potential toxicity on the organotypic tissue, Benostan cream was in contact with the tissue for the following time frames during the growth period: Days 0 to 12, Days 0 to 6, Days 7 to 12, Days 7 and 8, Days 9 and 10 and Day 11 and 12. On days when tissues were not being treated with the Benostan cream, only standard tissue growth media was used. After the 12 days of growth was completed the organotypic tissues were harvested for morphological examination using H&E staining as a visual subjective measure of the effect of Benostan cream on organotypic tissue growth.

HPV-16 Infectivity Inhibition Assay: An infectivity assay was performed as described by Biryukov and colleagues (Biryukov et al. (2015) Methods in Molecular Biology, 1249:317-331). Using the contact time of 7 to 12 days determined in the toxicity evaluation, three dilutions of Benostan cream were prepared (1:5, 1:10, and 1:15), and their effect on HPV-16 replication in organotypic tissue was measured.

As above, the Benostan cream was diluted with tissue culture media 1:5, 1:10, and 1:15, and these solutions were used in place of normal tissue culture media. Treatment exposures were for 7 to 12 days with a total growth time of 12 days. Controls of PBS diluted 1:5 with tissue culture media to mimic the dilution of the Benostan cream was used, as well as tissue culture media only. All measurements of HPV-16 genome replication and virus titers were compared to control tissue treated with PBS diluted with tissue culture growth media. After 12 days of growth the organotypic tissues were harvested and H&E staining was performed to assure tissue growth and HPV-16 genomes were measured per raft where total viral genomes and virions were measured.

### Results

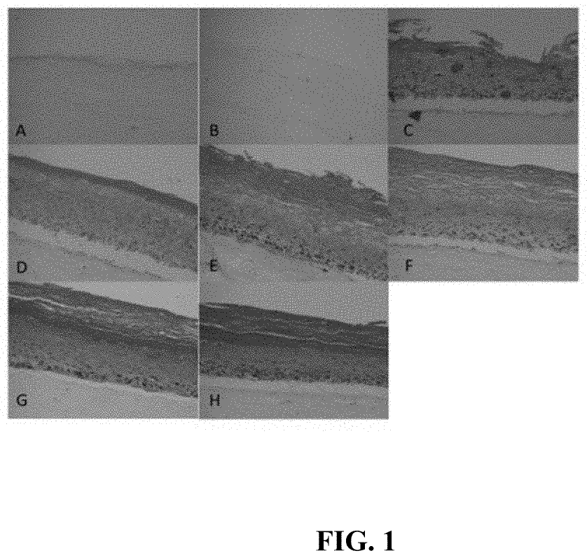

Evaluation of Tissue Toxicity: After the 12 days of growth was completed, the organotypic tissues were harvested for morphological examination using H&E staining as a visual subjective measure of the effect of the compound on organotypic tissue growth. Representative photographs on the toxicity testing are shown in FIG. 1.

It was clear that treatment of the tissue with the Benostan cream diluted 1:5 for 0 to 12 days and 0 to 6 days showed extreme toxicity as there was a complete lack of tissue growth following these exposure times (FIG. 1A-1B). All other treatment time periods (FIG. 1C-1F), while showing low levels of toxicity, were able to grow full-thickness epithelial tissues resembling the control tissues (FIG. 1G-1H). The longest time period of treatment, 7 to 12 days, that still allowed for full tissue growth with minimum toxicity (FIG. 1C) was chosen to test the effect of the Benostan cream on HPV-16 replication.

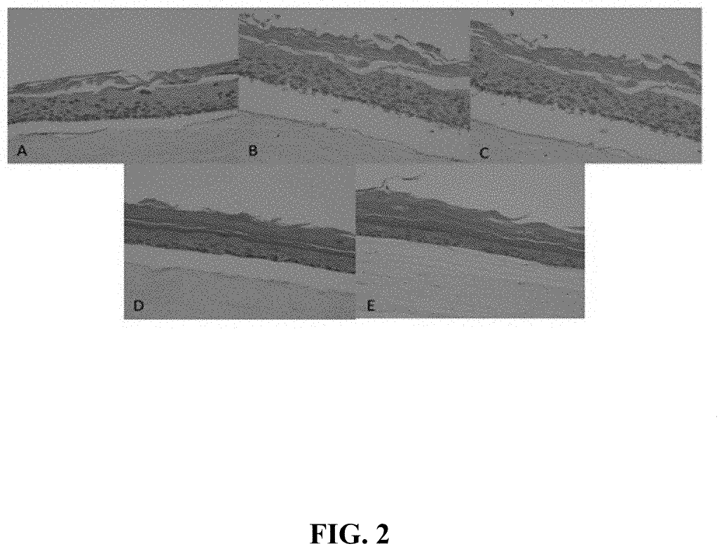

Evaluation of the Effects of Benostan Cream on HPV-16 Replication: Following 12 days of growth, the organotypic tissues were harvested and H&E staining was performed to assure tissue growth and HPV-16 genomes were measured per raft, where total viral genomes and virions were measured. As seen in FIG. 2, epithelial tissue grew under all treatment conditions.

Table 1 shows measurements of the effect of the Benostan cream on HPV-16 genome viral titer and replication. All three dilutions of the Benostan cream showed an approximately 40% to 50% decrease in viral titer (See Table 1). However, only the 1:15 dilution of the Benostan cream showed a decrease in total viral genomes, which was 34% (See Table 1).

TABLE 1
Effects of Benostan on HPV16 Genome Replication and Viral Titers

| | | TITER/ | % | TOTAL | % TOTAL |
|---|---|---|---|---|---|
| DRUG | DILUTION | RAFT | TITER | GENOMES | GENOMES |
| Benostan | 1:5 | 9 × 10³ | 64% | 8.6 × 10⁴ | 95% |
| Benostan | 1:10 | 7.2 × 10³ | 51% | 9 × 10⁴ | 99% |
| Benostan | 1:15 | 7.8 × 10³ | 56% | 6 × 10⁴ | 66% |
| E-media + | — | 1.4 × 10⁴ | — | 9.1 × 10⁴ | — |
| PBS | | | | | |
| E-media | — | 1.5 × 10⁴ | — | 7 × 10⁴ | — |
| only | | | | | |

### Discussion

These results described above show efficacy for the Benostan cream to inhibit the replication and viral production in epithelial tissue. A decrease in viral titers suggests a decrease in virus spread while a decrease in virus genomes suggests a decrease in persistence.

### REFERENCE STATEMENT

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

What is claimed is:

1. A pharmaceutical composition for the prevention or treatment of human papilloma virus (HPV)-associated disease in a subject in need thereof, comprising a pharmaceutically acceptable carrier and active agents comprising: Saint John's Wort, olive oil, and thyme; wherein the active agents are each present in therapeutically effective amounts to prevent HPV-associated disease in the subject.

2. The pharmaceutical composition of claim 1, comprising a pharmaceutically acceptable carrier and active agents comprising: Saint John's Wort, olive oil, and thyme; wherein the active agents are each present in therapeutically effective amounts to inhibit viral replication and viral production of HPV 16.

3. The pharmaceutical composition of claim 1, wherein the Saint John's Wort is in an amount of about 0.2 mg±1.5 mg, the olive oil is in an amount of about 1.0 mg±1.5 mg, and the thyme is in an amount of about 0.2 mg±1.5 mg.

4. The pharmaceutical composition of claim 1, wherein the pharmaceutical composition is formulated as a vaginal cream, ointment, or gel.

5. A method for the prevention or treatment of human papilloma virus (HPV)-associated disease in a subject in need thereof, comprising administering to the subject a pharmaceutical composition comprising a pharmaceutically acceptable carrier and active agents comprising: Saint John's Wort, olive oil, and thyme; wherein the active agents are each present in therapeutically effective amounts to prevent or treat HPV-associated disease in the subject.

6. The method of claim 5, comprising a pharmaceutically acceptable carrier and active agents comprising: Saint John's Wort, olive oil, and thyme; wherein the active agents are each present in therapeutically effective amounts to inhibit viral replication and viral production of HPV 16.

7. The method of claim 5, the Saint John's Wort is in an amount of about 0.2 mg±1.5 mg, the olive oil is in an amount of about 1.0 mg±1.5 mg, and the thyme is in an amount of about 0.2 mg±1.5 mg.

8. The method of claim 5, wherein the pharmaceutical composition is formulated as a vaginal cream, ointment, or gel.

9. The method of claim 5, wherein administration of the pharmaceutical composition is initiated after the subject's menses, is administered daily for a month followed by administration every other day for 2 to 6 months, further wherein the administration of the pharmaceutical composition is paused during the subject's menses.

10. A kit for the prevention or treatment of human papilloma virus (HPV)-associated disease in a subject in need thereof, comprising a pharmaceutical composition and instructions for the administration of the pharmaceutical composition in a manner effective to treat HPV-associated disease in the subject, wherein the pharmaceutical composition comprises a pharmaceutically acceptable carrier and active agents comprising: Saint John's Wort, olive oil, and thyme; wherein the active agents are each present in therapeutically effective amounts to prevent or treat HPV-associated disease in the subject.

11. The kit of claim 10, comprising a pharmaceutical composition and instructions for the administration of the pharmaceutical composition in a manner effective to inhibit viral replication and viral production of HPV 16 in the subject, wherein the pharmaceutical composition comprises a pharmaceutically acceptable carrier and active agents comprising: Saint John's Wort, olive oil, and thyme; wherein the active agents are each present in therapeutically effective amounts to inhibit viral replication and viral production of HPV 16.

12. The kit of claim 10, wherein the Saint John's Wort is in an amount of about 0.2 mg±1.5 mg, the olive oil is in an amount of about 1.0 mg±1.5 mg, and the thyme is in an amount of about 0.2 mg±1.5 mg.

13. The kit of claim 10, wherein the pharmaceutical composition is formulated as a vaginal cream, ointment, or gel.

14. The kit of claim 10, wherein the instructions for the administration of the pharmaceutical composition describe initiating administration of the pharmaceutical composition after the subject's menses, administering the pharmaceutical composition daily for a month followed by administration every other day for 2 to 6 months, and further wherein the administration of the pharmaceutical composition is paused during the subject's menses.
